SEQUENCE LISTING (5 sequences)

SEQ ID NO: 1 - DNA, 587 bp, Mus musculus
acttctgattctgacagactcaggaagaaaccatggtgctctctggggaagacaaaagcaacatcaaggctgcctgggggaagattggtggccatggtgctgaatatggagctgaagccctggaaaggatgtttgctagcttccccaccaccaagacctacttccctcactttgatgtaagccacggctctgcccaggtcaagggtcacggcaagaaggtcgccgatgctctggccaatgctgcaggccacctcgatgacctgcccggtgccctgtctgctctgagcgacctgcatgcccacaagctgcgtgtggatcccgtcaacttcaagctcctgagccactgcctgctggtgaccttggctagccaccaccctgccgatttcacccccgcggtgcatgcctctctggataaattccttgcctctgtgagcaccgtgctgacctccaagtaccgttaagctgccttctgcggggcttgccttctggccatgcccttcttctctcccttgcacctgtacctcttggtctttgaataaagcctgagtaggaagaagcctgcaaaaaaaaaaaaaaaaaaaaaaaaa

SEQ ID NO: 2 - DNA, 25 bp, Artificial sequence
  Probe sequence for mus musculus alpha globin
tttttttgcaagccccgcagaaggt

SEQ ID NO: 3 - DNA, 23 bp, Artificial sequence
  Probe sequence for mus musculus alpha globin
ttatttagagaagaagggcatgg

SEQ ID NO: 4 - DNA, 23 bp, Artificial sequence
  Probe sequence for mus musculus alpha globin
tttttaccaagaggtacaggtgc

SEQ ID NO: 5 - DNA, 22 bp, Artificial sequence
  Probe for mus musculus alpha globin
ttttttctacucaggctuuaut

(19) United States
(12) Patent Application Publication
(10) Pub. No.: US 2019/0300856 A1
(43) Pub. Date: Oct. 3, 2019

(54) Messenger RNA based expression of opsins and reporter proteins for electrophysiologic characterization of in vitro neurons and cardiomyocytes

(71) Applicant: Georgia Tech Research Corporation, Atlanta, GA (US)

(72) Inventors: Philip Santangelo, Atlanta, GA (US); Jonathan Kirschman, Atlanta, GA (US); Daryll Vanover, Atlanta, GA (US); Kevin Lindsay, Rome, GA (US)

(21) Appl. No.: 15/998,754

(22) Filed: Aug. 16, 2018

Related U.S. Application Data
(60) Provisional application No. 62/546,139, filed on Aug. 16, 2017.

(51) Int. Cl. C12N 5/077; C12N 15/85; C12N 5/0793; G01N 33/50; G01N 21/17

(52) U.S. Cl. CPC C12N 5/0657; C12N 15/85; C12N 5/0619; G01N 2021/1791; G01N 21/17; C12N 2015/859; G01N 33/5058

(57) ABSTRACT

Synthetic oligonucleotides are used to express opsins in cells, such as neurons and cardiomyocytes, for rapid induction of light-responsive electrophysiological behavior. Such induction can change the electrical properties of the cell. MEA analysis and use of voltage indicator proteins are described. Methods of testing drugs for their effect on electrical properties of the cell are described, along with methods for screening drugs that have an effect on a cell's electrical properties.

### CROSS-REFERENCE TO RELATED APPLICATIONS

The present application claims priority to U.S. Provisional Patent Application No. 62/546,139, filed on Aug. 16, 2017, which application is hereby incorporated by reference in its entirety.

### SEQUENCE LISTING

The instant application contains a Sequence Listing which has been submitted in ASCII format via paper and is hereby incorporated by reference in its entirety. Said ASCII copy, created on Aug. 16, 2018, is named GTRC7534sequence_ST25.txt and is 2,081 bytes in size.

### FIELD OF THE INVENTION

Provided herein are synthetic oligonucleotides to express opsins in cells, such as neurons and cardiomyocytes, for rapid induction of light-responsive electrophysiological behavior. Also provided are methods of using such synthetic oligonucleotides to give cells, such as neurons and cardiomyocytes, the capability to undergo rapid induction of light-responsive electrophysiological behavior.

### BACKGROUND OF THE INVENTION

Microelectrodes and microelectrode array (MEA) devices are used for biological research, as well as screening of drug compounds, which pertain to cardiac or neurological disorders. An MEA device uses electrodes to measure the field potential of nearby cells. Changes in field potential, such as spiking activity in neurons and cardiomyocytes, can be detected in an amplitude and location-specific manner depending on the pickup electrode.

Electrical activity is the primary readout in these experiments, which can be measured by an electrode or by imaging if a reporter protein is expressed.

Opsins are light-responsive ion channels which can be used for the control of action potentials in cells that exhibit electrical activity. They are currently in use for the scientific study of brain and cardiac electrical function (Entcheva, E. (2013) Cardiac optogenetics. American Journal of Physiology-Heart and Circulatory Physiology, 304, H1179-H1191; Ambrosi, C. M. and Entcheva, E. (2014) In Radisic, M. and Black Hi, L. D. (eds.), Cardiac Tissue Engineering: Methods and Protocols. Springer New York, New York, N.Y., pp. 215-228; and Whitmire, C. J., Waiblinger, C., Schwarz, C. and Stanley, G. B. (2016) Information Coding Through Adaptive Gating of Synchronized Thalamic Bursting. Cell reports, 14, 795-807.)

The current use of opsins, such as to generate model systems for testing new drugs and therapeutics, is limited by the features in currently used expression vectors. Adeno-associated virus (MV) is the primary vector used to express opsins. (Lin, J.Y. (2012) Optogenetic excitation of neurons with channelrhodopsins. Progress in Brain Research, 196, 29-47; Mattis, J., Tye, K. M., Ferenczi, E. A., Ramakrishnan, C., O'Shea, D. J., Prakash, R., Gunaydin, L. A., Hyun, M., Fenno, L. E., Gradinaru, V. et al. (2012) Principles for applying optogenetic tools derived from direct comparative analysis of microbial opsins. Nature methods, 9, 159-172.) However an individual AAV used has various limitations, such as permanence of expression, the induction of a strong innate and adaptive immune response, possible integration into the genome, and difficulty in controlling localization.

There is a need to provide for additional expression vectors to express opsin without the above limitations, drawbacks and risks of AAV vectors.

### SUMMARY OF THE INVENTION

As specified in the Background Section, there is a great need in the art to develop expression vectors suitable for opsin expression that do not have the drawbacks and limitations of AAV vectors.

In one aspect is provided an mRNA expression vector for the expression of opsin, wherein the mRNA expression vector comprises a nucleic acid sequence encoding opsin. The mRNA expression vector comprises one or more modified nucleosides selected from 2-thiouridine, 5-methyl cytidine, and pseudouridine. The mRNA expression vector may further comprise an enzymatic m7G on the 5′ triphosphate end. The mRNA expression vector is purified by HPLC chromatography. The mRNA expression vector is complexed with protamine, packaged in a liposome, or packaged in a nanoparticle. The mRNA expression vector may comprise a heterologous promoter, such as a T7 promoter. The mRNA expression vector may comprise a Kozak sequence. In some embodiments, the opsin is an excitatory opsin. In some embodiments, the opsin is an inhibitory opsin. In some embodiments, the excitatory opsin is Channel-rhodopsin II (ChR2) or calcium translocating channelrhodopsin (CatCh). In some embodiments, the inhibitory opsin is JAWS.

In various embodiments, the mRNA can also be modified as known in the art to prevent or reduce premature degradation.

In another aspect is provided a cell that comprises an mRNA expression vector wherein the mRNA expression vector comprises a nucleic acid sequence encoding opsin. In some embodiments, the cell is a cardiac cell. In some embodiments, the cell is a neuronal cell.

In another aspect is provided a method for transiently expressing an opsin in a cell comprising introducing an mRNA expression vector for the expression of opsin into the cell. In some embodiments, the introducing step comprises transiently transfecting the cell with the mRNA expression vector. In some embodiments, the mRNA expression vector comprises a nucleic acid sequence encoding opsin. In some embodiments, the opsin is an excitatory opsin. In some embodiments, the opsin is an inhibitory opsin. In some embodiments, the transiently expressed opsin can be detected for 1 to 7 days post-transfection. In some embodiments, the transiently expressed opsin can be detected from 3 hours post transfection to 7 days post-transfection. In some embodiments, the mRNA expression vector is purified before transient transfection by high pressure liquid chromatography (HPLC).

In another aspect is provided a method for measuring electrical activity in a cell in response to light stimulation. The method comprises (a) transiently transfecting the mRNA expression vector of claim 1 into the cell, (b) stimulating the cell with light, and (c) measuring the field potential in the cell. In some embodiments, measuring the field potential comprises performing multi-electrode array (MEA) analysis on the cell. In some embodiments, the cell is stimulated with pulsed light. In some embodiments, MEA analysis comprises measuring synchronization events between pulsed light stimulation and electrical activity from the cell. In yet another aspect is method for testing a drug comprising contacting a cell with the drug and measuring electrical activity in the cell. The cell may be a cardiac cell and the method comprises measuring the beat rate of the cardiac cell in response to pacing the cardiac cell at a beat rate using light stimulation. The method may comprise transiently transfecting an mRNA expression vector comprising a voltage indicator protein.

These and other aspects of the present invention will be apparent to those of ordinary skill in the art in the following description, claims and drawings.

### BRIEF DESCRIPTION OF THE DRAWINGS

The accompanying Figures, which are incorporated in and constitute a part of this specification, illustrate several aspects described below. The patent or application file contains at least one drawing executed in color. Copies of this patent or patent application publication with color drawing(s) will be provided by the Office upon request and payment of the necessary fee.

FIG. 1 shows Opsin expression in Hek293 cells 16 hours post-transfection with Channel Rhodopsin 2 (ChR2)-YFP mRNA. The live cells were imaged on a widefield microscope using a YFP filter set and 10x/NA 0.25 objective.

FIG. 2 shows staining of each of GFP, NeuN, DAPI and a merged image (of GFP, NeuN and DAPI staining) in rat neuronal cultures transfected with GFP mRNA. NeuN shows the nuclei of neurons, with GFP visibly expressed in neurons.

FIG. 3 shows a screenshot of ChR2 functional testing in rat cortical neuronal cultures with multi-electrode array (MEA) on an Axion Maestro-Lumos system.

FIG. 4 shows a graph of response rate versus time post-transfection of ChR-2 transfected neurons. Synchronization of ChR-2 transfected neuron action potentials and excitation. Response rates are shown along with SD for 3 wells per condition.

FIG. 5 shows the response of ChR2-transfected neurons up to 144 hours post-transfection with ChR2 mRNA. The Y-axis shows the percentage of wells with ChR2-transfected neurons that respond to excitation. The X-axis shows samples with various numbers of hours (24, 28, 42, 96, 120 and 144) post-transfection.

FIG. 6 shows a plot of an excitation intensity comparison between ChR2 and calcium translocating channelrhodopsin (CatCh) mRNA-transfected neonatal rat ventricular myocytes (NRVMs). The Y-axis shows the percentage of LED power and the X-axis shows the time post-transfection for each of ChR2- and CatCh mRNA-transfected NRVMs.

FIG. 7 shows a plot of the maximum beat rate comparison between ChR2 and CatCh mRNA-transfected NRVMs. The Y-axis shows the maximum driven frequency and the X-axis shows the number of hours post-transfection.

FIG. 8 shows a CatCh-imaging time course In NRVMs. CatCh-transfected NRVMs were fixed at varying time points and stained for alpha-sarcomeric actinin and V5-tagged CatCh protein. Images were obtained using a Zeiss Plan-Apo 63×1.4 NA oil objective on an UltraVIEW Spinning Disk Confocal Microscope.

FIG. 9 shows two graphs on CatCh flow expression in NRVMs. CatCh-transected NRVMs were processed and stained stained for viability, α-SA, and CatCh-V53. Cells were measured using a BD LSRFortessa flow cytometer and results were analyzed in FlowJo.

FIG. 10 shows strength Duration curves demonstrating the higher sensitivity of CatCh over Channelrhodopsin 2 for NRVM stimulation.

FIG. 11 shows the rates and thresholds of NRVM capture over time.

FIG. 12 shows sample action potential signals from a single NRVM cell transfected with FlicR RNA.

### DETAILED DESCRIPTION OF THE INVENTION

As specified in the Background Section, there is a great need in the art to develop expression vectors suitable for opsin expression that do not have the drawbacks and limitations of AAV vector's.

Unless defined otherwise, technical and scientific terms used herein have the same meaning as commonly understood by one of ordinary skill in the art to which this invention belongs.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The terms “a,” “an,” and “the” do not denote a limitation of quantity, but rather denote the presence of “at least one” of the referenced item.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J. Additional techniques are explained, e.g., in U.S. Pat. No. 7,912,698 and U.S. patent appl. Pub. Nos. 2011/0202322 and 2011/0307437.

In one aspect is provided an mRNA expression vector for the expression of opsin wherein the mRNA expression vector comprises a nucleic acid sequence encoding opsin. The mRNA expression vector may comprise a heterologous promoter, such as a T7 promoter. The mRNA expression vector may comprise a Kozak sequence. Those of ordinary skill appreciate that any promoter or regulatory sequence may be chosen to provide suitable expression of the opsin. Without wishing to be bound by theory, the inventors have found that mRNA provides a superior way to express opsin in a cell for further testing (such as on the electrical activity of a cell in response to light stimulation). Opsin can be expressed by the mRNA expression vector in as little as three hours, with expression able to persist for seven days. The various modes of mRNA expression described have minimal impact on the normal functioning of a cell, for example by not introducing a virus into the cell.

In some embodiments, the opsin is an excitatory opsin. In some embodiments, the opsin is an inhibitory opsin. Examples of excitatory opsins include, but are not limited to, Channel-rhodopsin II (ChR2) and CatCh, which is activated with pulsed blue light. Examples of inhibitory opsins include, but are not limited to, JAWS, which is activated by constant orange or red light. (Chuong, A. S., Miri, M. L., Busskamp, V., Matthews, G. A. C., Acker, L. C., Sorensen, A. T., Young, A., Klapoetke, N. C., Henninger, M. A., Kodandaramaiah, S. B. et al. (2014) Noninvasive optical inhibition with a red-shifted microbial rhodopsin. Nat Neurosci, 17, 1123-1129.)

In some embodiments, the mRNA expression vector is capped. For example, the mRNA is capped by enzymatic m7G capping of the 5′ triphosphate end. Such capping may reduce the ability of retinoic acid-inducible gene I (RIG-I), or other members of the RIG-I-like receptor (RLR) protein family, to sense the mRNA and provoke an undesirable innate immune response. For example, without capping at the 5′ triphosphate end, RIG-I could rapidly sense and degrade the mRNA. With such capping, the mRNA expression vector can evade detection by RIG-I and other members of the RLR protein family. In various embodiments, capping of the 5′ triphosphate end of the mRNA expression vector is effective for the mRNA expression vector to express the opsin protein for at least 3 days, at least 4 days, at least 5 days, and at least 6 days after transfection into a cell comprising RIG-I or a member of the RLR protein family.

In some embodiments, modified nucleosides are incorporated into the mRNA, wherein incorporation of the modified nucleosides is effective to reduce RNA-dependent protein kinase (PKR) and 2′-5′-oligoadenylate synthetase (OAS) activation. The modified nucleoside can include one or more of 2-thiouridine, 5-methyl cytidine (5meC), and pseudouridine (e.g., N1-methyl pseudouridine). In some embodiments, the mRNA comprises both 5meC and pseudouridine. In some embodiments, the modified nucleoside is diaminopurine (DAP), N6-methyl-2-aminoadenosine (me6DAP), N6-methyladenosine (me6A), 5-carboxycytidine (5caC), 5-formylcytidine (5fC), 5-hydroxycytidine (5haC), 5-hydroxymethylcytidine (5hmC), 5-methoxycytidine (5maC), 5-methylcytidine (5meC), N4-methylcytidine (me4C), thienoguanosine (tyG), 5-carboxymethylesteruridine (5camU), 5-formyluridine (5fU), 5-hydroxymethyluridine (5hmU), 5-methoxyuridine (5moU), or 5-methyluridine (5meU).

In some embodiments, the mRNA expression vector is purified by high pressure liquid chromatography (HPLC). Purification, such as by HPLC, may allow for one or both of a reduction in immune activation, an increase in translational potential, and a reduction in TLR signaling in cell culture. In some embodiments, the mRNA expression vector comprises both 5meC and pseudouridine and is HPLC-purified.

In some embodiments, the mRNA expression vector is complexed with protamine, a small arginine-rich nuclear protein that can stabilize mRNA. mRNA complexed with protamine may be taken up by a cell via endocytosis. In some embodiments, the mRNA expression vector is packaged in a liposome. The liposome may be a nanoliposome. In some embodiments, the mRNA expression vector is packaged in a nanoparticle. The nanoparticle may be a lipid nanoparticle.

In another aspect is provided a cell that comprises an mRNA expression vector wherein the mRNA expression vector comprises a nucleic acid sequence encoding opsin. In some embodiments, the cell is a cardiac cell. In some embodiments, the cell is a neuronal cell. In some embodiments, the cell is an HEK 293 cell. In some embodiments, the cell comprises a second mRNA expression vector comprising a nucleic acid sequence encoding a voltage indicator protein, such as FlicR.

In another aspect is provided a method for transiently expressing an opsin in a cell comprising introducing an mRNA expression vector for the expression of opsin into the cell. In some embodiments, the introducing step comprises transiently transfecting the cell with the mRNA expression vector. In some embodiments, the mRNA expression vector comprises a nucleic acid sequence encoding opsin. In some embodiments, the opsin is an excitatory opsin. In some embodiments, the opsin is an inhibitory opsin. In some embodiments, the opsin is Channel Rhodopsin 2, which is a blue light excitable ion channel which allows for controlled firing of action potentials in cells which exhibit electrical activity. Other examples of opsins include, but are not limited to, Type I opsins (e.g., bacteriorhodopsin, xanthorhodopsin, halorhodopsin, rhodopsin I, rhodopsin B, channelrhodopsin (ChR), an archaerhodopsin (Arch), Type II opsins (e.g., ciliary opsins, pinopsin, rhodopsin (Rh1), long-wavelength sensitive (OPN1LW) opsin, middle-wavelength sensitive (OPN1MW) opsin, short-wavelength sensitive (OPN1SW) opsin, parapinospin, parietopsin, panopsin (OPN3), teleost multiple tissue (TMT) opsin, r-opsin, melanopsin, Go-opsin, RGR opsin, peropsin, and neuropsin.)

In some embodiments, a second mRNA expression vector comprising a nucleic acid sequence encoding a voltage indicator protein, such as FlicR, is transiently transfected into the cell. Other voltage indicator proteins that may be used include, but are not limited to, FlaSH, VSFP1, SPARC, VSFP2, Flare, VSFP3.1, Mermaid, hVOS, PROPS, ArcLight, Arch, ElectricPk, VSFP-Butterfly, VSFP-CR, Mermaid2, Mac GEVI, QuasAr1, QuasAr2, Archer, ASAP1, Ace GEVI, Pado, and ASAP2f.

In some embodiments, transient transfection comprises electroporation. In some embodiments, transient transfection comprises lipofection. In some embodiments, transient transfection comprises modified PEI-mediated delivery, such as JET-PEI. In some embodiments, transient transfection comprises complexing the mRNA with a virus-like polymer (e.g., Viromer® Red). In some embodiments, lipofection is undertaken with modified mRNA (e.g., mRNA modified with 5meC and pseudouridine), which is effective to improve expression of transcripts from the mRNA in the cell.

In some embodiments, modified nucleosides are incorporated into the mRNA expression vector, wherein incorporation of the modified nucleosides is effective to reduce RNA-dependent protein kinase (PKR) and 2′-5′-oligoadenylate synthetase (OAS) activation. The modified nucleoside can include one or more of 2-thiouridine, 5-methyl cytidine (5meC), and pseudouridine (e.g., N1-methyl pseudouridine). In some embodiments, the mRNA comprises both 5meC and pseudouridine. In some embodiments, the modified nucleoside is diaminopurine (DAP), N6-methyl-2-aminoadenosine (me6DAP), N6-methyladenosine (me6A), 5-carboxycytidine (5caC), 5-formylcytidine (5fC), 5-hydroxycytidine (5haC), 5-hydroxymethylcytidine (5hmC), 5-methoxycytidine (5maC), 5-methylcytidine (5meC), N4-methylcytidine (me4C), thienoguanosine (tyG), 5-carboxymethylesteruridine (5camU), 5-formyluridine (5fU), 5-hydroxymethyluridine (5hmU), 5-methoxyuridine (5moU), or 5-methyluridine (5meU). In various embodiments, the mRNA expression vector localizes to the cellular membrane of the cell in which the mRNA expression vector is transiently expressed.

In some embodiments, the mRNA expression vector comprises a polyA tail. A polyA tail may be added to the mRNA expression vector. A polyA tail already present on in the mRNA expression vector may be increased in length. As a non-limiting example, the polyA tail may be enzymatically added. The inventors have found that increased expression of a protein encoded by the mRNA expression vector with a polyA tail of at least 100 nucleotides was unexpected, particularly because those of ordinary skill in the art consider hyperadenylation to be an unwanted feature correlated with decay of mRNA. See, e.g., Sokoloski K. J., et al., Virus-mediated mRNA decay by hyperadenylation. Genome Biology. 2009; 10(8):234. doi:10.1186/gb-2009-10-8-234. The polyA tail may range from 75 to 1200 bases in length. The polyA tail may be from 75-100 bases in length, 85-110 bases in length, 100-125 bases in length, 110-135 bases in length, 125-150 bases in length, 135-160 bases in length, 150-175 bases in length, 170-220 bases in length, 175-225 bases in length, 200-250 bases in length, 225-275 bases in length, 250-300 bases in length, 275-325 bases in length, 300-350 bases in length, 325-375 bases in length, 350-400 bases in length, 375-425 bases in length, 400-450 bases in length, 425-475 bases in length, 450-500 bases in length, 475-525 bases in length, 500-550 bases in length, 550-600 bases in length, 600-650 bases in length, 650-700 bases in length, 700-750 bases in length, 750-800 bases in length, 800-850 bases in length, 850-900 bases in length, 900-950 bases in length, 950-1000 bases in length, 1000-1050 bases in length,1050-1100 bases in length, 1100-1150 bases in length, or 1150-1200 bases in length.

In some embodiments, the transiently expressed opsin can generally be detected for 1 to 7 days post-transfection, e.g., 1 to 2 days, 2 to 4 days, 3 to 5 days, 4 to 6 days and 5 to 7 days post-transfection.

In some embodiments, the mRNA expression vector is purified before transient transfection. Purification may be performed by high pressure liquid chromatography (HPLC). Purification, such as by HPLC, may allow for one or both of a reduction in immune activation, an increase in translational potential, and a reduction in TLR signaling in cell culture.

In another aspect is provided a method for functionally characterizing a cell expressing any of the mRNA expression vectors for the expression of opsin described herein. The method may comprise performing multi-electrode array (MEA) analysis on the cell expressing the mRNA expression vector. MEA analysis can also be performed on a similar cell not expressing the mRNA expression vector as a control. MEA analysis may comprise detection of field potential, for example spiking activity in a cell. The cell may be a cardiac cell. The cell may be a neural cell. MEA analysis may be performed on a device that can simultaneously measure and provide light excitation to samples in multiple compartments, such as a Maestro® device from Axion Biosystems that allows for simultaneous measurement and light excitation of 16 electrodes per well in a 48 well plate.

Further, when spontaneous activity occurs in both cortical primary cells (e.g., from a rat) and neonatal rat ventricular myocytes (NRVMs), the functional expression of an opsin (e.g., Channel rhodopsin 2 (ChR2)) can be measured as a synchronization event between electrical activity and pulsed light excitation from LEDs embedded in the MEA device (e.g., an Axion Lumos device).

In another aspect is provided a method for screening drugs and/or testing one or more drugs for their effect on functional characterization of a cell expressing an opsin. The drug may be applied to a cardiac cell or a neural cell. The drug screening may be used to find drugs that are candidates for treating a cardiac disorder or a neural disorder. The method may comprise performing multi-electrode array (MEA) analysis on a cell expressing the mRNA expression vector in which the drug for screening or testing is applied. MEA analysis can also be performed on a similar cell expressing the mRNA expression vector but without the drug applied. MEA analysis may comprise detection of field potential, for example spiking activity in a cell. MEA analysis may be performed on a device that can simultaneously measure and provide light excitation to samples in multiple compartments, such as a Maestro® device from Axion Biosystems that allows for simultaneous measurement and light excitation of 16 electrodes per well in a 48 well plate. The functional expression of an opsin (e.g., ChR2) can be measured as a synchronization event between electrical activity and pulsed light excitation from LEDs embedded in the MEA device. If the cell is a cardiac cell, MEA analysis can comprise testing the beat rate or the electrical response in a quantitative manner. For example, the cardiac cells expressing the mRNA expression vector can be paced at various beat rates such as 1 Hz, 2 Hz or 3Hz. Also, different light patterns from the MEA device can be used to simulate an arrhythmia or another anomaly.

In some embodiments, the method is for screening drugs and/or testing one or more drugs for their effect on arrhythmia in a cardiac cell expressing an opsin. Without wishing to be bound by theory, numerous pharmacotherapies suffer from arrhythmogenic side effects. Assessment of whether a drug may increase or exacerbate arrhythmia is needed for at least the reason that over 1% of patients prescribed class III antiarrhythmic agents suffer from Torsades de Pointes, a highly fatal condition. A better understanding of which drugs are risky for such patients can reduce fatalities.

The method can provide for current methods of studying the properties of primary or stem cell-derived cardiomyocytes in a real-time longitudinal study. The method may comprise transfecting into a cardiac cell an opsin and a voltage indicator protein (e.g., FlicR). The opsins may convert visible light into an electrical response and the voltage indicator protein can exhibit a detectable change in fluorescent intensity in response to a voltage change. Transfection of both the voltage indicator protein and the opsin can be undertaken with a modified PEI, such as Viromer Red. The localization of the expressed proteins may be assayed by microscopy and/or flow cytometry.

In various embodiments, the function of transfected transfected cells is evaluated. The transfected cell may be exposed to a range of optical intensities of light that can stimulate the opsin. For example, CatCh may be stimulated using 475 nm blue light. Various stimulation durations may be used, from 0.5 to 30 milliseconds, from 1 to 15 milliseconds, from 1 to 5 milliseconds, from 4 to 10 milliseconds, from 10 to 15 milliseconds, or from 15 to 30 milliseconds. Monophasic square waves can be used in a train at various frequencies.

The cells may also be imaged in a swept field microscope to assay for the signal produced by the voltage regulator protein, e.g., FlicR.

Additional Embodiments:

1. An mRNA expression vector comprising a nucleic acid sequence encoding opsin.

2. The mRNA expression vector of embodiment 1, wherein the opsin is an excitatory opsin or an inhibitory opsin.

3. The mRNA expression vector of embodiment 2, wherein the excitatory opsin is Channel-rhodopsin II (ChR2).

4. The mRNA expression vector of embodiment 2, wherein the inhibitory opsin is JAWS.

5. A cell comprising the mRNA vector of any one of embodiments 1-4.

6. The cell of embodiment 5, wherein the cell is a cardiac cell or a neuronal cell.

7. A method for transiently expressing an opsin in a cell, the method comprising transiently transfecting the mRNA expression vector of any of embodiments 1-4 into the cell.

8. The method of embodiment 7, wherein the mRNA expression vector comprises a nucleic acid sequence encoding opsin.

9. The method of embodiment 7 or embodiment 8, wherein the transiently expressed opsin can generally be detected for 1 to 7 days post-transfection.

10. The method of any one of embodiments 7 to 9, wherein the mRNA expression vector is purified before transient transfection by high pressure liquid chromatography (HPLC).

11. A method for measuring electrical activity in a cell in response to light stimulation, the method comprising:

transiently transfecting the mRNA expression vector of any of embodiments 1-4 into the cell;

stimulating the cell with light;

measuring the field potential in the cell.

12. The method of embodiment 11, wherein measuring the field potential comprises performing multi-electrode array (MEA) analysis on the cell.

13. The method of embodiment 11 or embodiment 12, further comprising measuring the field potential in a second cell not transfected with the mRNA expression vector.

14. The method of any one of embodiments 10-13, wherein the cell is a cardiac cell or a neural cell.

15. The method of any one of embodiments 12-14, wherein the MEA analysis is performed on a device that can simultaneously stimulate the cell with light and measure the field potential in the cell.

16. The method of any one of embodiments 12-15, wherein the cell is stimulated with pulsed light.

17. The method of embodiment 16, wherein MEA analysis comprises measuring synchronization events between pulsed light stimulation and electrical activity from the cell.

18. A method for testing a drug comprising

contacting a cell with the drug, and measuring electrical activity in the cell according to any of embodiments 12-17.

19. The method of embodiment 18, wherein the cell is a cardiac cell and wherein the method comprises measuring the beat rate of the cardiac cell in response to pacing the cardiac cell at a beat rate using light stimulation.

20. The method of embodiment 18 or embodiment 19, wherein the voltage indicator protein is FlicR.

21. The method of any one of embodiments 18-20, comprising transfecting an opsin and a voltage indicator protein (e.g., FlicR) into the cell.

22. The method of any one of embodiments 18-21, wherein transfecting comprises packaging the voltage indicator protein and/or the opsin with a modified PEI, such as Viromer Red.

23. The method of any one of embodiments 18-22, comprising imaging the transfected cells in a swept field microscope to assay for a signal produced by the voltage regulator protein.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

Example 1: Synthesis and labeling of an mRNA for in vitro transfection

All IVT mRNAs were synthesized by Moderna Therapeutics (Boston, MA, USA) containing identical sequences and included 5′ capping and polyadenylation. EGFP-encoding mRNAs either were synthesized without modified nucleosides or with total incorporation of 5meC and Pseudouridine. RNA was stored frozen in −80° C. and subjected to minimal freeze-thaw cycles. A detailed protocol for MTRIPs assembly and characterization was described in Santangelo et al., Probes for intracellular RNA imaging in live cells. Methods in enzymology, 2012, 505: 383. Four oligos complementary to four adjacent sequences spanning the mouse alpha globin 3′ UTR (NM_001083955.1, sequence: ACTTCTGATTCTGACAGACTCAGGAAGAAACCATGGTGCT CTCTGGGGAAGACAAAAGCAACATCAAGGCTGCCTGGGGGAAGATTGGTGGCCATGGTG CTGAATATGGAGCTGAAGCCCTGGAAAGGATGTTTGCTAGCTTCCCCACCACCAAGACCTA CTTCCCTCACTTTGATGTAAGCCACGGCTCTGCCCAGGTCAAGGGTCACGGCAAGAAGGT CGCCGATGCTCTGGCCAATGCTGCAGGCCACCTCGATGACCTGCCCGGTGCCCTGTCTG CTCTGAGCGACCTGCATGCCCACAAGCTGCGTGTGGATCCCGTCAACTTCAAGCTCCTGA GCCACTGCCTGCTGGTGACCTTGGCTAGCCACCACCCTGCCGATTTCACCCCCGCGGTGC ATG CCTCTCTGGATAAATTCCTTGCCTCTGTGAGCACCGTGCTGACCTCCAAGTACCGTTA AGCTGCCTTCTGCGGGGCTTGCCTTCTGGCCATGCCCTTCTTCTCTCCCTTGCACCTGTAC CTCTTGGTCTTTGAATAAAGCCTGAGTAGGAAGAAGCCTGCAAAAAAAAAAAAAAAAAAAAA AAAA (SEQ ID NO: 1)) of the IVT mRNA were generated.

Sequences were adjacent due to the small length of the UTR region. Probe sequences were as follows:

(SEQ ID NO: 2)
Biotin-T(C6-Amino)-TTTTT-T(C6-Amino)-G-C-A-A-G-C-
C-C-C-G-C-A-G-A-A-G-G-T(C6-Amino)
(SEQ ID NO: 3)
Biotin-T(C6-Amino)-TTATT-T(C6-Amino)-A-G-A-G-A-A-
G-A-A-G-G-G-C-A-T(C6-Amino)-G-G
(SEQ ID NO: 4)
Biotin-T(C6-Amino)-TTTT-T(C6-Amino)-A-C-C-A-A-G-
A-G-G-T(C6-Amino)-A-C-A-G-G-T(C6-Amino)-G-C
(SEQ ID NO: 5)
Biotin-T(C6-Amino)-TTTTTT-C-T(C6-Amino)-A-C-U-C-
A-G-G-C-T(C6-Amino)-U-U-A-U-T(C6-Amino)-C

Each sequence was analyzed via nucleotide BLAST to ensure minimal off-target binding. Sequences were purchased as 2′-O-methyl RNA-DNA chimeric oligonucleotides 17-18 bases long with a short 5-7 poly(T) linker and 4 C6-amino-modified thymidines. The oligos included a 5′ biotin modification and were purchased from Biosearch Technologies (Petaluma, Calif., USA). The oligonucleotides were labeled with Cy3b-NHS ester (GE Healthcare) or Dylight 650/680-NHS esters (Pierce) using manufacturer protocols. MTRIPs were assembled by incubation with Neutravidin (Pierce) for 1 hour at RT followed by filtration using 30 kD MWCO centrifugal filters (Millipore). mRNA was buffer exchanged into 1×PBS, heated to 70° C. for 10 min and immediately placed on ice, combined with MTRIPs in a 1:1 mRNA:MTRIP ratio and then incubated overnight at 37° C. The next day, the labeled mRNA was filtered using a 200 kD MWCO ultrafiltration unit (Advantec MFS Inc.) and concentrated by 50 kD MWCO centrifugal filters (Millipore). Alternative filters tested during protocol optimization included 100 and 300 kD MWCO, but either did not filter unbound MTR1Ps successfully or failed to successfully retain mRNA.

Example 2: Development and verification of mRNA expressing an opsin

In order to develop a platform for opsin expression using NT mRNA, the inventors first generated mRNA corresponding to the sequence for Channel Rhodopsin II (ChR2), a blue light excitable ion channel which allows the controlled firing of action potentials in cells which exhibit electrical activity. ChR2 was initially chosen due to its widespread use in optogenetics studies. The coding region was codon optimized and embedded it in the same 5′ and 3′ UTR cassette including the mouse alpha globin 3′ UTR as used in Example 1. mRNA was formulated with the PEI-derivative Viromer Red.

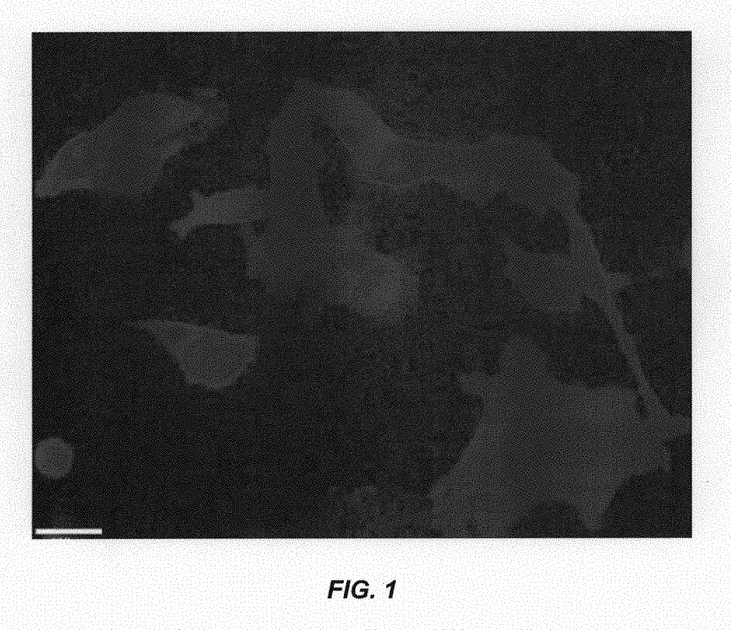

Following the IVT process, Hek293 cells were transfected to ensure that the protein expressed and was localized to the cellular membrane (FIG. 1) in a similar manner to that shown using pDNA transfection by Lin et al (4). FIG. 1 shows Opsin expression in Hek293 cells 16 hours post-transfection with ChR2-YFP mRNA, with imaging performed ion a widefield microscope using a YFP filter set and 10×/NA 0.25 objective.

Example 3: Functional validation of expressed opsins in neuronal cells

Functional validation of the expressed ChR2 protein was performed. In order to show that mRNA-based expression was possible in neurons, primary rat cortical neural cells obtained from E18 Embryonic rat cortex were transfected using GFP-mRNA and Viromer Red. Using antibody staining with a nuclear marker which is neuron specific, NeuN, it was observed that some cells contained both GFP fluorescence and NeuN nuclear staining, which indicated that rat cortical neurons were transfected.

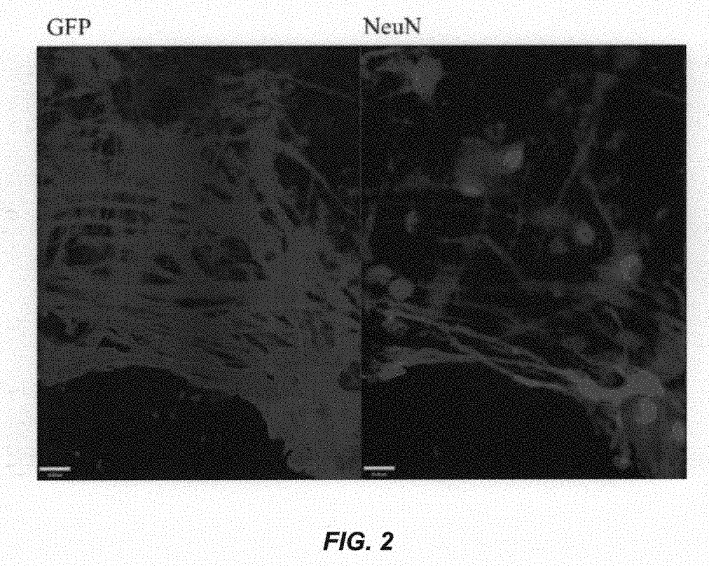

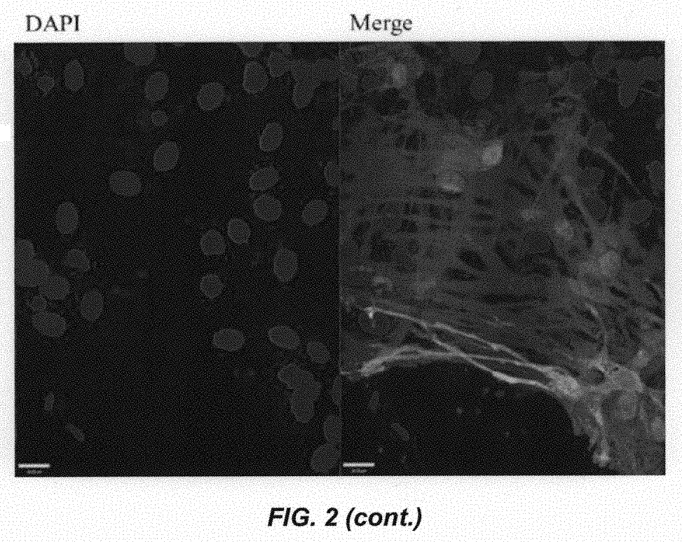

The data is shown in FIG. 2. GFP expression colocalizes with the neuron-specific nuclear marker NeuN in mixed rat cortical neuronal cultures transfected with GFP mRNA. NeuN (red) marks the nuclei of neurons. GFP is visibly expressed in neurons, though other cell types are visible and may or may not express GFP. Cells were fixed and stained at 24 hours post-transfection and imaged with a 40×1.2 NA objective on an Ultraview Spinning Disk microscope.

A functional assay using a multi-electode array (MEA) on an Axion Maestro-Lumos system was performed. The Axion Maestro-Lumos system has 16 electrode readouts per well multiplexed to a 48 well simultaneous measurement format, with fully controllable and synchronized 4-color LED excitation per well. The MEA approach is particularly suited to testing opsin function because although it is single-cell sensitive, it has the capability of broad spatial detection using multiple electrodes as well as excellent throughput.

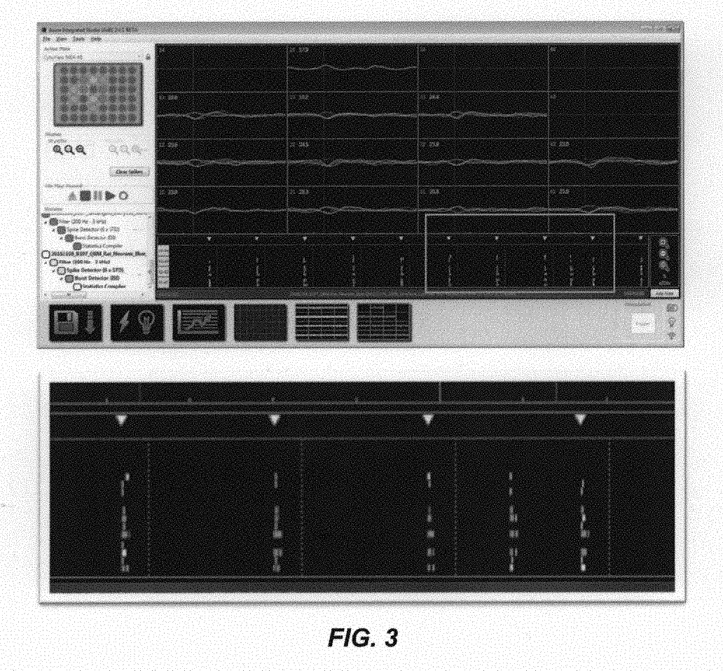

An example of an MEA reading of neurons expressing ChR2 and responding to blue pulsed excitation light is depicted in FIG. 3, with the screen displays waveforms detected in 16 electrodes in a single well of a 48 well plate. The blowup in FIG. 3 shows arrows which represent pulsed blue light. White and red dots indicate detected action potentials synchronized with the expression. Red dots in particular indicate neuron burst activity. Spontaneous firing activity also occurs without stimulus.

Rat cortical neurons were transfected three weeks post-plating on an MEA plate with ChR2 mRNA using 2000 ng, 4000 ng, and 6000 ng of mRNA while the same delivery vehicle to mRNA ratio was maintained. In order to address issues of excitation absorption by the YFP tag, mRNA encoding ChR2 with a V5 epitope tag sequence was included. The electrical response to LED stimulation was measured from 24 to 144 hours post-transfection. At 24 hours post-transfection, wells in all replicates showed action potential stimulation synchronized with blue light excitation pulses.

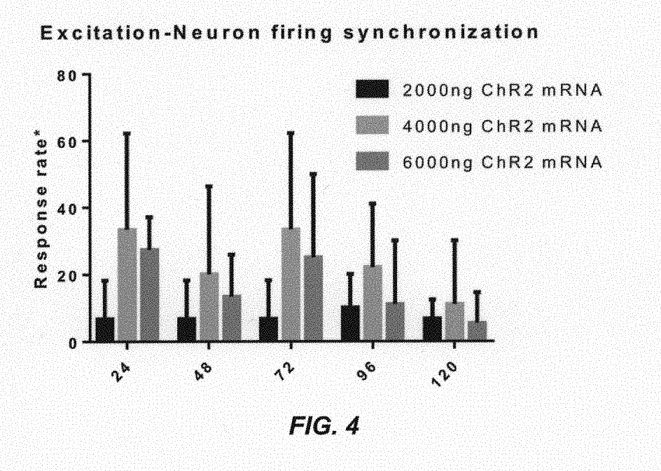

The data is shown in FIG. 4, which indicates the synchronization of ChR-2 transfected neuron action potentials and excitation. The response rate is calculated by the number of time-synchronized action potentials corresponding to an excitation pulse. An excitation of 100% light pulsed for 5 ms was used to stimulate action potentials, with a 3 second delay between pulses. Response rates are shown along with SD for 3 wells per condition. Three amounts of mRNA were used in order to find an optimal delivery amount. 2 out of 3 wells responded to excitation at 48 and 72 hours post-transfection, with complete lack of response at 144 hours. This indicated that the opsin has a relatively long half-life. The synchronization of excitation and neuronal firing were also calculated.

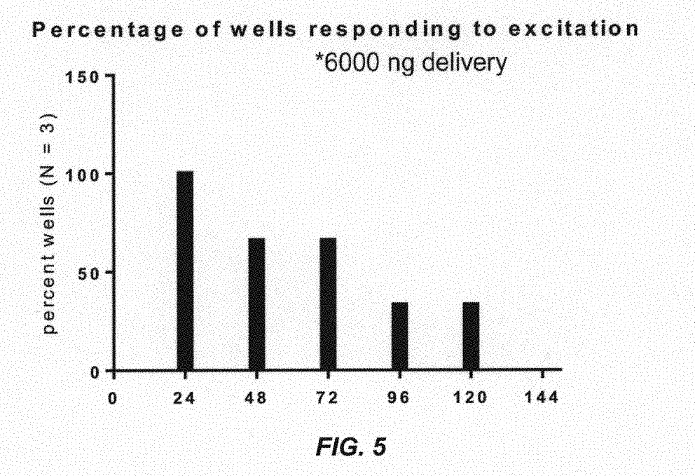

It was found that the response rate, the percent of excitation signals which resulted in a synchronized neuronal network firing event, was best with 4000ng of mRNA. FIG. 5 shows the response of ChR2-transfected neurons up to 144 hours post-transfection with ChR2 mRNA. Rat cortical neuronal cells were transfected with 6000 ng of ChR2 mRNA and assayed via MEA daily until no response was detected from excitation light at 6 days post transfection. The percentage of transfected wells (N=3) responding are plotted at each time point is indicated in the figure.

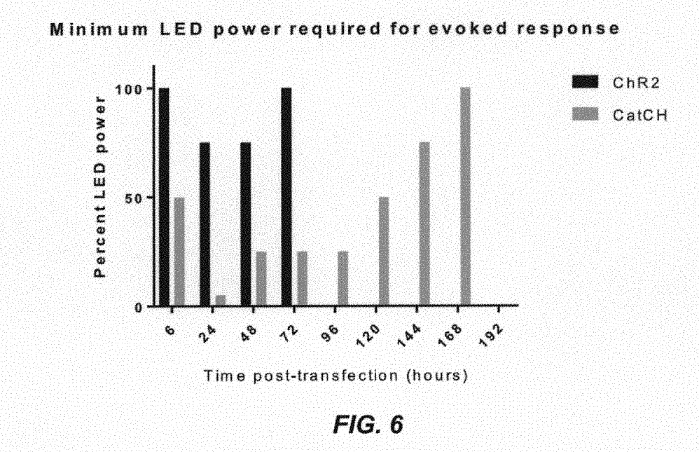

The percent of wells which responded to light at each time point is shown in FIG. 6, which represents an excitation intensity comparison between ChR2 and CatCh mRNA-transfected NRVMs. NRVMs were transfected one day after plating on MEA plates with either ChR2 or CatCh mRNA. The sensitivity of the opsin to excitation light is inversely proportional to the percent LED power required to evoke a response. CatCh shows the highest sensitivity at 24 hours post transfection, where it responds to 5% intensity LED light. This is 15 times the LED power required for ChR2.

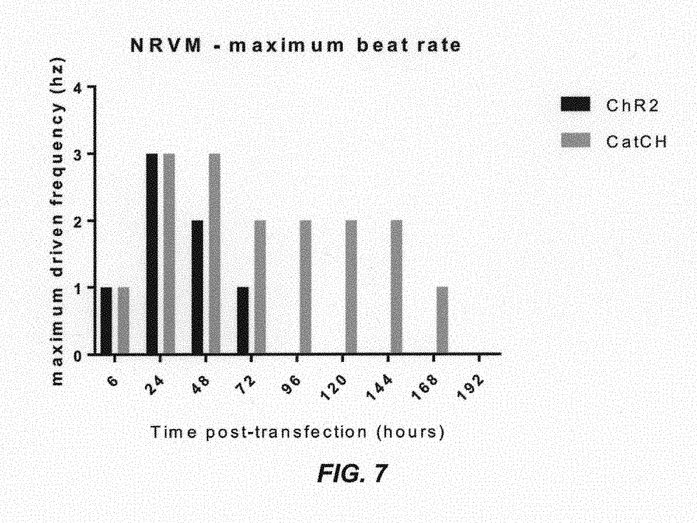

Furthermore, excitation rates of 1 hz, 2hz, and 3hz were applied to CatCh- and ChR2-transfected cells. CatCh-transfected cells were able to be driven at higher beat rates (3hz) up to 72 hours post- transfection compared to ChR2 transfected cells. The data are shown in FIG. 7.

FIG. 7 shows a maximum beat rate comparison between ChR2 and CatCh mRNA-transfected NRVMs. NRVMs were transfected one day after plating on MEA plates with either ChR2 or CatCh mRNA. They were driven at 1 Hz, 2 Hz, and 3 Hz via LED pulsing. Responses were recorded if all three replicate wells were able to beat at the indicated rates using maximum intensity excitation light. CatCh was able to sustain higher frequencies throughout the time course experiment.

As the amount of mRNA and structure of the opsin are very similar, this can be inferred as due to enhance sensitivity that reflects how fewer opsin molecules per cell were necessary to drive cardiac action potentials. When opsin expression is low, such as at timepoints several days following transfection, cardiac cells could only be driven at the slower rate of 1 hz successfully.

The methodology of RNA based expression can allow rapid production of any opsin and can be used for comparisons of new opsins as they emerge, which may be difficult to control using viral expression vectors.

Example 4: Evaluating CatCh Expression in Neonatal Rat Ventricular Cardiomyocytes (NRVMs):

Two protein classes expressed in this study are opsins that convert visible light into an electrical response and voltage indicators that change in fluorescent intensity in response to voltage changes. These proteins are used extensively in the field of neuroscience. However, their use for controlling the excitability and studying the phenotypes of different cardiomyocyte platforms has not been fully explored. The inventors have demonstrated the use of IVT mRNA to optically control and measure the electrical activity of cardiomyocytes, which can allow for study of different cardiomyocyte platforms and their response to drugs.

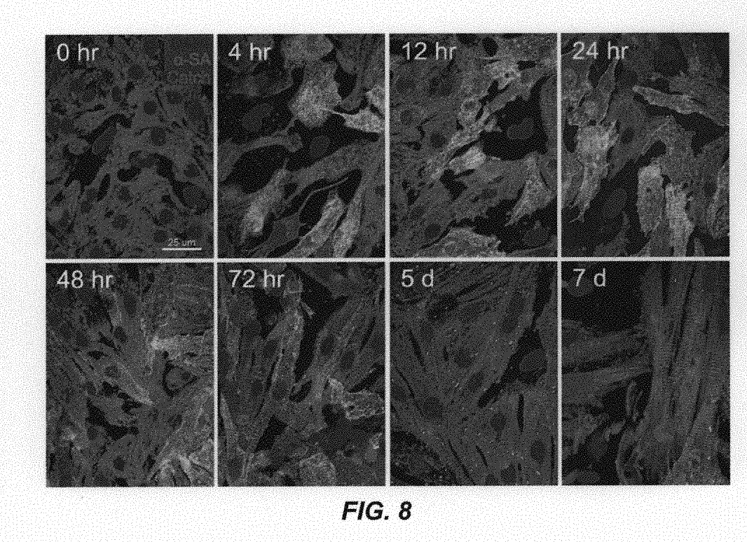

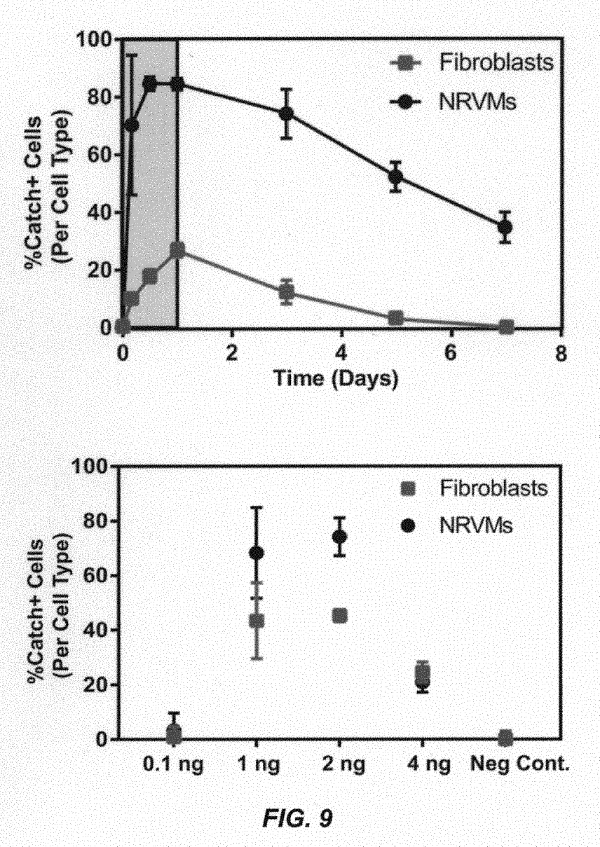

Transfectlon of the NRVMs using mRNA was demonstrated. Transfection conditions were varied using GFP mRNA. After identifying the optimal vehicle for transfection (Viromer Red), the expression of the protein of interest, CatCh-V5, was assessed using a 24 hour pulse of mRNA. The data are shown in FIGS. 8 and 9.

From the imaging time course (FIG. 8), strong CatCh-V5 expression is achieved in NRVMS, as identified by expression of alpha-SA (a marker of cardiomyocytes). In the time course, CatCh-transfected NRVMs were fixed at varying time points and stained for alpha-sarcomeric actinin and V5-tagged CatCh protein. Images were obtained using a Zeiss Plan-Apo 63×1.4 NA oil objective on an UltraVlEW Spinning Disk Confocal Microscope.

CatCh-transected NRVMs were processed and stained stained for viability, α-SA, and CatCh-V53. Cells were measured using a BD LSRFortessa flow cytometer and results were analyzed in FlowJo. CatCh-V5 localizes to the membrane of transfected cells by roughly 12 hours after transfection. Expression peaks at 24 hours, dropping off significantly by 5 days. The flow analysis, shown in FIG. 9, indicates efficient transfection of NRVMs over fibroblasts in culture, and the time course profile matches what was seen in the images.

Example 5: Evaluating CatCh Function in NRVMs

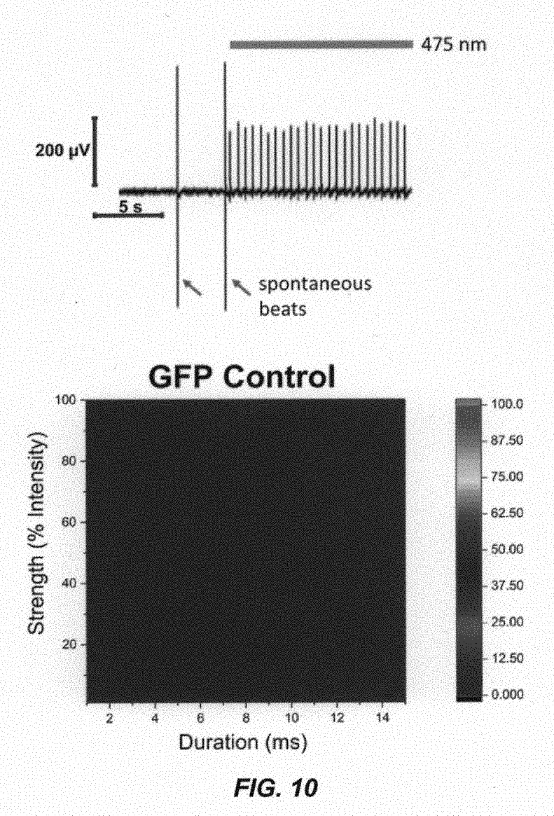

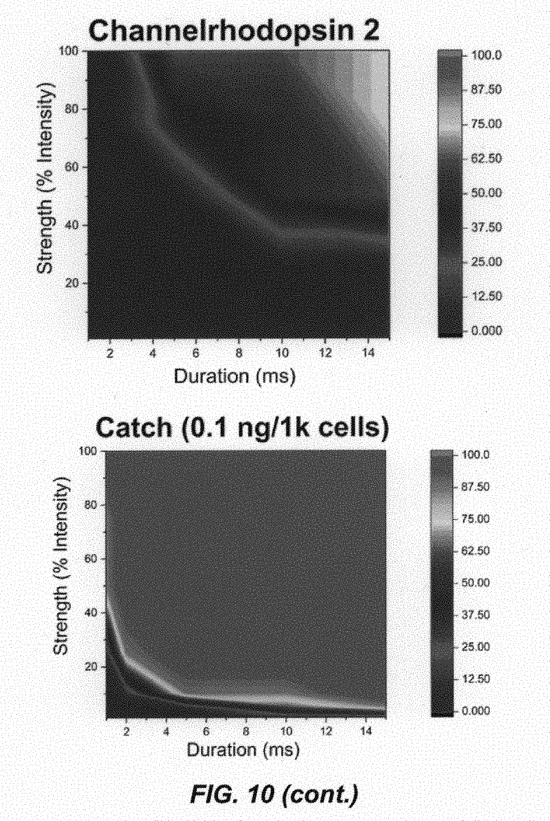

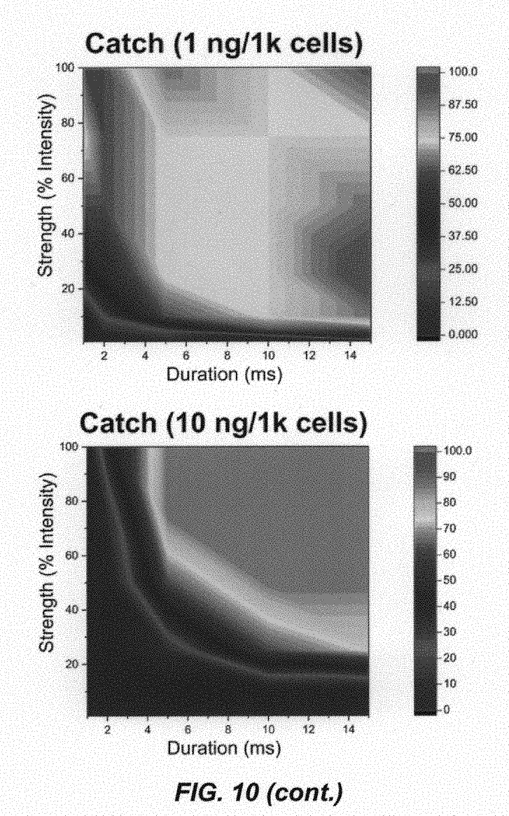

In this example, the function of transfected NRVMs was evaluated. CatCh is an opsin, which induces a membrane depolarization in response to blue light stimulation. CatCh transfected NRVMs were exposed to a range of optical intensities (0 to 100% power) using 475 nm blue LED light. At each light intensity, we scanned a range of stimulation durations (1 to 15 milliseconds). A train of monophasic square waves at a frequency of 2 Hz for a total of 30 seconds was utilized. The capture rate of CatCh was compared to that of Channelshodopsin 2, a commonly used opsin. The results are shown in FIG. 10.

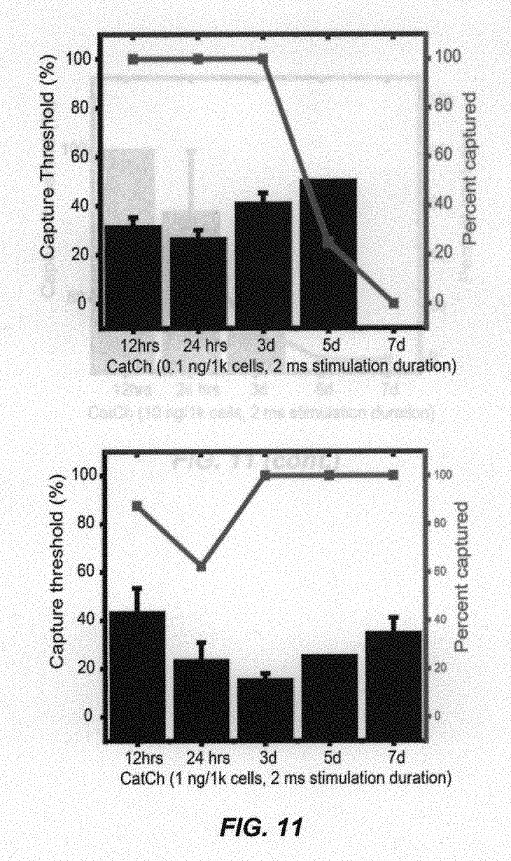

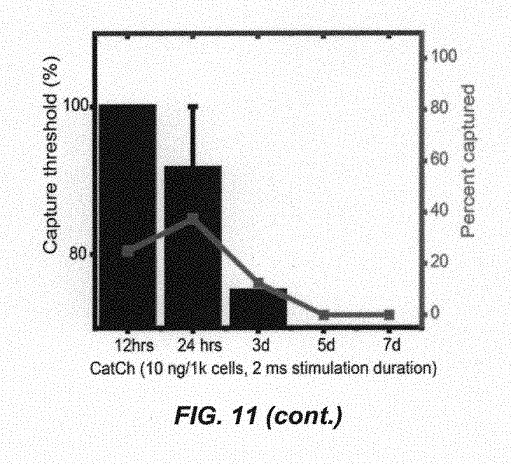

CatCh demonstrates superior sensitivity, resulting in a higher percentage of captured cells at lower light intensity and a shorter pulse duration. Cells were then subjected to blue light stimulation and analyzed for capture at different timepoints. FIG. 11 shows the results of this experiment, which indicates that 1 ng/1,000 cells is the optimal RNA dose to achieve reliable pacing over time.

Example 6: Optical Action Potential Characterization

The ability to use light to characterize the electrical properties of the NRVMs was demonstrated. FlicR is a voltage indicator protein that fluoresces at different intensities depending on the electrical potential of the cell membrane. Cells were transfected with mRNA encoding FlicR so as to assess the characteristics of the cells' action potentials on a large number of cells using microscopy.

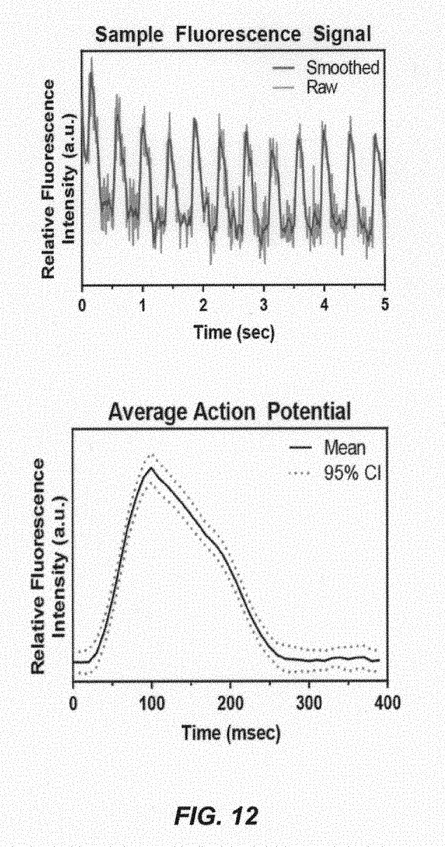

NRVMs were transfected with mRNA encoding FlicR and imaged them on a swept field microscope in live culture. A series of images were obtained at 100 Hz and brightness over time measurements were performed in a region of interest per cell. The signal was processed in a custom MATLAB script. FIG. 12 shows a sample plot of a single cell's action potential over time and the average action potential captured for that cell.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

1. An mRNA expression vector comprising a nucleic acid sequence encoding opsin, wherein the mRNA expression vector comprises one or more modified nucleosides selected from 2-thiouridine, 5-methyl cytidine, and pseudouridine.

2. The mRNA expression vector of claim 1, wherein the mRNA expression vector further comprises an enzymatic m7G on the 5′ triphosphate end.

3. The mRNA expression vector of claim 2, wherein the mRNA expression vector is purified by HPLC chromatography.

4. The mRNA expression vector of claim 3, wherein the mRNA expression vector is complexed with protamine, packaged in a liposome, or packaged in a nanoparticle.

5. The mRNA expression vector of claim 1, wherein the opsin is an excitatory opsin or an inhibitory opsin.

6. The mRNA expression vector of claim 1, wherein the excitatory opsin is Channel-rhodopsin II (ChR2) or calcium translocating channelrhodopsin (CatCh).

7. The mRNA expression vector of claim 1, wherein the inhibitory opsin is JAWS.

8. A cell comprising the mRNA vector of claim 1.

9. The cell of claim 8, wherein the cell is a cardiac cell or a neuronal cell.

10. A method for transiently expressing an opsin in a cell, the method comprising transiently transfecting the mRNA expression vector of claim 1 into the cell.

11. The method of claim 10, wherein the mRNA expression vector comprises a nucleic acid sequence encoding opsin.

12. The method of claim 10, wherein the transiently expressed opsin can be detected for 1 to 7 days post-transfection.

13. The method of claim 10, wherein the mRNA expression vector is purified before transient transfection by high pressure liquid chromatography (HPLC).

14. A method for measuring electrical activity in a cell in response to light stimulation, the method comprising:
transiently transfecting the mRNA expression vector of claim 1 into the cell;
stimulating the cell with light; and
measuring the field potential in the cell.

15. The method of claim 14, wherein measuring the field potential comprises performing multi-electrode array (MEA) analysis on the cell.

16. The method of claim 14, wherein the cell is stimulated with pulsed light.

17. The method of claim 16, wherein MEA analysis comprises measuring synchronization events between pulsed light stimulation and electrical activity from the cell.

18. A method for testing a drug comprising contacting a cell with the drug, and measuring electrical activity in the cell according to claim 14.

19. The method of claim 18, wherein the cell is a cardiac cell and wherein the method comprises measuring the beat rate of the cardiac cell in response to pacing the cardiac cell at a beat rate using light stimulation.

20. The method of claim 18, further comprising transiently transfecting an mRNA expression vector comprising a voltage indicator protein.
